(19) United States
(12) Patent Application Publication
(10) Pub. No.: US 2007/0010487 A1
(43) Pub. Date: Jan. 11, 2007

(54) Chemotherapeutic formulations of zosuquidar trihydrochloride and modified cyclodextrins

(76) Inventors: Jeff Schwegman, Bloomington, IN (US); Mark Edgar, Rancho Santa Fe, CA (US); Branimir Sikic, Stanford, CA (US); Daniel Hoth, San Francisco, CA (US); David Socks, Carlsbad, CA (US); Scott Glenn, La Jolla, CA (US); John Marcelletti, San Diego, CA (US); Michael J. Walsh, San Diego, CA (US); Pratik S. Multani, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/418,400

(22) Filed: May 3, 2006

Related U.S. Application Data
(60) Provisional application No. 60/696,939, filed on Jul. 6, 2005.
(60) Provisional application No. 60/696,756, filed on Jul. 6, 2005.
(60) Provisional application No. 60/696,930, filed on Jul. 6, 2005.

(51) Int. Cl. A61K 31/724

(52) U.S. Cl. 514/58

(57) ABSTRACT

The present invention relates to a method of treating patients with leukemias, solid tumors, and other malignancies using chemotherapeutic agents in combination with zosuquidar that has been solubilized by a modified cyclodextrin, such as sulfobutylcyclodextrin or hydroxypropyl cyclodextrin. The invention is also directed to pharmaceutical formulations comprising zosuquidar in combination with a modified cyclodextrin.

### RELATED APPLICATION

This application claims priority under 35 U.S.C. § 119(e) to U.S. Provisional Application No. 60/696,939 filed Jul. 6, 2005, U.S. Provisional Application No. 60/696,756 filed Jul. 6, 2005, and U.S. Provisional Application No. 60/696,930 filed Jul. 6, 2005 which are incorporated by reference herein in their entirety, and which are hereby made a part of this specification.

### FIELD OF THE INVENTION

The present invention relates to a method of treating patients with leukemias, solid tumors, and other malignancies using chemotherapeutic agents in combination with zosuquidar that has been solubilized by a modified cyclodextrin, such as sulfobutylcyclodextrin or hydroxypropyl cyclodextrin. The invention is also directed to pharmaceutical formulations comprising zosuquidar in combination with a modified

### BACKGROUND OF THE INVENTION

The field of oncology is in the midst of a major evolution. In the past, the treatment of cancer has been dominated by empiric, “one-size-fits-all” treatments based on types and stages of tumors. Toxic chemotherapy drugs have dominated the treatment landscape despite a very low cure rate, particularly against the most common cancers and those with known metastatic disease.

Now, treatments in development are targeted against specific proteins. Such targeting is based on a more robust knowledge of cancer mechanisms, which often crosses over many tumor types. These treatments are designed to work in defined subsets of patients, typically based on expression and function of the target protein rather than the type of tumor, and often in combination with standard chemotherapies. Advances in the molecular analysis of cancers will enable the identification of such susbsets of patients and the coupling of targeted therapeutics to novel diagnostic approaches.

The future of oncology lies in defining the disease in molecular terms (i.e., genetics, genomics, proteomics) and tailoring therapies according to individual tumor and normal cell properties. This new paradigm will predetermine likely responders, assess responses earlier, and adjust treatment based on continued molecular analyses of tumors.

Drug resistance is one of the most difficult problems that must be overcome in order to achieve successful treatment of human tumors with chemotherapy. Clinically, drug resistance, a characteristic of intrinsically resistant tumors (for example, colon, renal, and pancreas) or other malignancies, may be evident at the onset of therapy. Alternatively, acquired drug resistance results when tumors or malignancies initially respond to therapy but become refractory to subsequent treatments. Once a tumor or malignancy has acquired resistance to a specific chemotherapeutic agent, it is common to observe collateral resistance to other structurally similar agents.

Multidrug resistance (MDR), the ability of cancer cells to become resistant to the agent(s) actively used for therapy, as well as other drugs that are structurally and functionally unrelated, is a particularly insidious form of drug resistance. Zosuquidar, a 10,11-methanobenzosuberane derivative, is useful in enhancing the efficacy of existing cancer chemotherapeutics and for treating multidrug resistance. However, zosuquidar has limited solubility in aqueous solution, such that the formulation concentration is limited, resulting in a large number of vials to contain doses in the potentially efficacious range (e.g., a clinical formulation of zosuquidar without solubility enhancers of 50 mg per 30 mL vial that requires 11 units to provide 550 mg of zosuquidar).

### SUMMARY OF THE INVENTION

Dosage forms and treatment regimens for treating solid tumors, leukemias such as acute myelogenous leukemia (AML) and other malignancies that result in increased rates of complete remission and increased cancer-free survival rates are desirable. Also desirable are intravenous zosuquidar formulations having a greater zosuquidar concentration and increased content per dosage unit. Zosuquidar formulated with a modified cyclodextrin to enhance its solubility provides an improved formulation that can offer such advantages. Hydroxypropylcyclodextrins and sulfobutylcyclodextrins are particularly preferred modified cyclodextrins for use in zosuquidar formulations.

Accordingly, in a first aspect a stable chemotherapeutic composition comprising zosuquidar in combination with a modified cyclodextrin is provided.

In an embodiment of the first aspect, the modified cyclodextrin is a hydroxypropyl-β-cyclodextrin.

In an embodiment of the first aspect, the modified cyclodextrin is a sulfobutylcyclodextrin, e.g., a polyanionic β-cyclodextrin derivative with a sodium sulfonate salt separated from a lipophilic cavity by a butyl ether spacer group.

In an embodiment of the first aspect, the composition is in lyophilized form.

In an embodiment of the first aspect, the composition is in solution form, e.g., dextrose solution.

In an embodiment of the first aspect, the stable chemotherapeutic composition is in liquid unit dosage form, comprising from about 10 mg/mL to about 30 mg/mL zosuquidar and from about 100 mg/mL to about 200 mg/mL sulfobutylcyclodextrin.

In an embodiment of the first aspect, the stable chemotherapeutic composition is in liquid unit dosage form, comprising from about 20 mg/mL to about 25 mg/mL zosuquidar and from about 125 mg/mL to about 175 mg/mL sulfobutylcyclodextrin.

In an embodiment of the first aspect, the stable chemotherapeutic composition is in liquid unit dosage form, comprising about 22.5 mg/mL zosuquidar and about 150 mg/mL sulfobutylcyclodextrin.

In an embodiment of the first aspect, the stable chemotherapeutic composition is in lyophilized form, comprising zosuquidar and sulfobutylcyclodextrin in a weight ratio of zosuquidar to sulfobutylcyclodextrin of from about 1:5.7 to about 1:7.4.

In an embodiment of the first aspect, the stable chemotherapeutic composition is in lyophilized form, comprising zosuquidar and sulfobutylcyclodextrin in a weight ratio of zosuquidar to sulfobutylcyclodextrin of from about 1:6 to about 1:7.

In an embodiment of the first aspect, the stable chemotherapeutic composition is in lyophilized form, comprising zosuquidar and sulfobutylcyclodextrin in a weight ratio of zosuquidar to sulfobutylcyclodextrin of about 1:6.73.

In a second aspect, a pharmaceutical kit is provided, the kit comprising at least one container containing a stable chemotherapeutic composition comprising zosuquidar in combination with a modified cyclodextrin; and directions for administering the chemotherapeutic composition to treat a malignancy that expresses P-glycoprotein.

In an embodiment of the second aspect, the modified cyclodextrin is hydroxypropyl-β-cyclodextrin.

In an embodiment of the second aspect, the modified cyclodextrin is sulfobutylcyclodextrin.

In an embodiment of the second aspect, the malignancy is acute myelogenous leukemia

In an embodiment of the second aspect, the kit further comprises at least one container containing daunorubicin and at least one container containing cytarabine, and directions for administering the daunorubicin and cytarabine to treat newly diagnosed acute myelogenous leukemia.

In an embodiment of the second aspect, the kit further comprises at least one container containing Mylotarg, and directions for administering the Mylotarg to treat relapsed acute myelogenous leukemia.

In a third aspect, a pharmaceutical kit is provided, the kit comprising at least one vial containing a stable chemotherapeutic lyophilized composition, comprising about 275 mg/vial zosuquidar and about 1850 mg/vial sulfobutylcyclodextrin; and directions for reconstituting the lyophilized composition with a 15 mL of a 5% dextrose solution and administering the reconstituted solution to a patient to treat acute myelogenous leukemia.

In a fourth aspect, a method of treating cancer in a patient exhibiting positive P-glycoprotein expression or positive P-glycoprotein function is provided, the method comprising administering to the patient a chemotherapeutic agent that is a substrate for P-glycoprotein efflux and a stable chemotherapeutic composition comprising zosuquidar in combination with a modified cyclodextrin, whereby the cancer is treated.

In an embodiment of the fourth aspect, the modified cyclodextrin is a hydroxypropyl-β-cyclodextrin.

In an embodiment of the fourth aspect, the modified cyclodextrin is a sulfobutylcyclodextrin, e.g., a polyanionic β-cyclodextrin derivative with a sodium sulfonate salt separated from a lipophilic cavity by a butyl ether spacer group.

In an embodiment of the fourth aspect, the stable chemotherapeutic composition is in lyophilized form.

In an embodiment of the fourth aspect, the stable chemotherapeutic composition is in solution form, e.g., a dextrose solution.

In an embodiment of the fourth aspect, the stable chemotherapeutic composition is in liquid unit dosage form, comprising from about 10 mg/mL to about 30 mg/mL zosuquidar and from about 100 mg/mL to about 200 mg/mL sulfobutylcyclodextrin.

In an embodiment of the fourth aspect, the stable chemotherapeutic composition is in liquid unit dosage form, comprising from about 20 mg/mL to about 25 mg/mL zosuquidar and from about 125 mg/mL to about 175 mg/mL sulfobutylcyclodextrin.

In an embodiment of the fourth aspect, the stable chemotherapeutic composition is in liquid unit dosage form, comprising about 22.5 mg/mL zosuquidar and about 150 mg/mL sulfobutylcyclodextrin.

In an embodiment of the fourth aspect, the stable chemotherapeutic composition is in lyophilized form, comprising zosuquidar and sulfobutylcyclodextrin in a weight ratio of zosuquidar to sulfobutylcyclodextrin of from about 1:5.7 to about 1:7.4.

In an embodiment of the fourth aspect, the stable chemotherapeutic composition is in lyophilized form, comprising zosuquidar and sulfobutylcyclodextrin in a weight ratio of zosuquidar to sulfobutylcyclodextrin of from about 1:6 to about 1:7.

In an embodiment of the fourth aspect, the stable chemotherapeutic composition is in lyophilized form, comprising zosuquidar and sulfobutylcyclodextrin in a weight ratio of zosuquidar to sulfobutylcyclodextrin of about 1:6.73.

In an embodiment of the fourth aspect, the cancer is acute myelogenous leukemia.

In an embodiment of the fourth aspect, the cancer is a carcinoma (e.g., breast cancer or ovarian cancer), a sarcoma, or a hematologic malignancy (e.g., acute lymphoblastic leukemia, chronic myeloid leukemia, plasma cell dyscrasias, lymphoma, or myelodysplasia).

In an embodiment of the fourth aspect, the chemotherapeutic agent is an anthracycline (e.g., doxorubicin, daunorubicin, epirubicin, idarubicin, or mitoxantrone).

In an embodiment of the fourth aspect, the chemotherapeutic agent is a Topoisomerase-II inhibitor (e.g., etoposide or teniposide).

In an embodiment of the fourth aspect, the chemotherapeutic agent is a vinca (e.g., vincristine, vinblastine, vinorelbine, and vindesine).

In an embodiment of the fourth aspect, the chemotherapeutic agent is a taxane (paclitaxel or docetaxel).

In an embodiment of the fourth aspect, the chemotherapeutic agent is selected from the group consisting of gleevec, dactinomycin, bisantrene, mitoxantrone, actinomyocin D, mithomycin C, mitramycin, methotrexate, adriamycin, mitomycin, and mithramycin, anthracene, and epipodophyllo-toxin.

In an embodiment of the fourth aspect, the chemotherapeutic agent comprises daunorubicin and cytarabine, and the cancer is newly diagnosed acute myelogenous leukemia.

In an embodiment of the fourth aspect, the chemotherapeutic agent comprises Mylotarg, and the cancer is relapsed acute myelogenous leukemia.

In a fifth aspect, a method of administering a therapeutic agent that is a substrate for P-glycoprotein efflux to a patient in need thereof is provided, wherein the patient exhibits positive P-glycoprotein expression or P-glycoprotein function, the method comprising administering the therapeutic agent to the patient; and administering a stable P-glycoprotein efflux pump inhibiting composition comprising zosuquidar in combination with a modified cyclodextrin to the patient.

In an embodiment of the fifth aspect, the modified cyclodextrin is a hydroxypropyl-β-cyclodextrin.

In an embodiment of the fifth aspect, the modified cyclodextrin is a sulfobutylcyclodextrin, e.g., a polyanionic β-cyclodextrin derivative with a sodium sulfonate salt separated from a lipophilic cavity by a butyl ether spacer group.

In an embodiment of the fifth aspect, the stable chemotherapeutic composition is in lyophilized form.

In an embodiment of the fifth aspect, the stable chemotherapeutic composition is in solution form, e.g., a dextrose solution.

In an embodiment of the fifth aspect, the stable chemotherapeutic composition is in liquid unit dosage form, comprising from about 10 mg/mL to about 30 mg/mL zosuquidar and from about 100 mg/mL to about 200 mg/mL sulfobutylcyclodextrin.

In an embodiment of the fifth aspect, the stable chemotherapeutic composition is in liquid unit dosage form, comprising from about 20 mg/mL to about 25 mg/mL zosuquidar and from about 125 mg/mL to about 175 mg/mL sulfobutylcyclodextrin.

In an embodiment of the fifth aspect, the stable chemotherapeutic composition is in liquid unit dosage form, comprising about 22.5 mg/mL zosuquidar and about 150 mg/mL sulfobutylcyclodextrin.

In an embodiment of the fifth aspect, the stable chemotherapeutic composition is in lyophilized form, comprising zosuquidar and sulfobutylcyclodextrin in a weight ratio of zosuquidar to sulfobutylcyclodextrin of from about 1:5.7 to about 1:7.4.

In an embodiment of the fifth aspect, the stable chemotherapeutic composition is in lyophilized form, comprising zosuquidar and sulfobutylcyclodextrin in a weight ratio of zosuquidar to sulfobutylcyclodextrin of from about 1:6 to about 1:7.

In an embodiment of the fifth aspect, the stable chemotherapeutic composition is in lyophilized form, comprising zosuquidar and sulfobutylcyclodextrin in a weight ratio of zosuquidar to sulfobutylcyclodextrin of about 1:6.73.

In an embodiment of the fifth aspect, the stable chemotherapeutic composition is a dextrose solution.

In an embodiment of the fifth aspect, the therapeutic agent comprises an immunosuppressant (e.g., cyclosporine, cyclosporine A, or tacrolimus).

In an embodiment of the fifth aspect, the therapeutic agent comprises a steroid (e.g., dexamethasone, hydrocortisone, corticosterone, triamcinolone, aldosterone, or methylprednisolone).

In an embodiment of the fifth aspect, the therapeutic agent comprises an antiepileptic (e.g., phenytoin).

In an embodiment of the fifth aspect, the therapeutic agent comprises an antidepressant (e.g., citalopram, thioperidone, trazodone, trimipramine, amitriptyline, or phenothiazines).

In an embodiment of the fifth aspect, the therapeutic agent comprises an antipsychotic (e.g., fluphenazine, haloperidol, thioridazine, or trimipramine).

In an embodiment of the fifth aspect, the therapeutic agent comprises a protease inhibitor (e.g., amprenavir, indinavir, lopinavir, nelfinavir, ritonavir, or saquinavir).

In an embodiment of the fifth aspect, the therapeutic agent comprises a calcium blocker (e.g., bepridil, diltiazem, flunarizine, lomerizine, secoverine, tamolarizine, verapamil, nicardipine, prenylamine, or fendiline).

In an embodiment of the fifth aspect, the therapeutic agent comprises a cardiac drug (e.g., digoxin, diltiazem, verapamil, or talinolol).

In an embodiment of the fifth aspect, the therapeutic agent comprises daunorubicin and cytarabine, and the patient is newly diagnosed with acute myelogenous leukemia.

In an embodiment of the fifth aspect, the therapeutic agent comprises Mylotarg, and the patient is diagnosed with relapsed acute myelogenous leukemia.

### BRIEF DESCRIPTION OF THE DRAWINGS

FIG. 1 illustrates the increase of zosuquidar concentration in solution as a function of sulfobutylcyclodextrin concentration.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

Zosuquidar

U.S. Pat. Nos. 5,643,909 and 5,654,304 disclose a series of 10,11-methanobenzosuberane derivatives useful in enhancing the efficacy of existing cancer chemotherapeutics and for treating multidrug resistance. One such derivative having good activity, oral availability, and stability, is zosuquidar, a compound of formula (2R)-anti-5-3-[4-(10,11-difluoromethanodibenzosuber-5-yl)piperazin-1-yl]-2-hydroxypropoxy)quinoline.

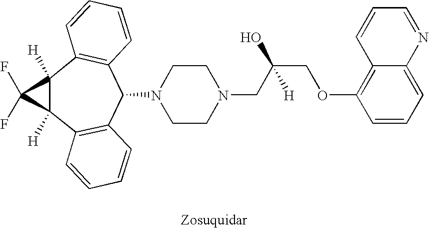

Given the limitations of previous generations of MDR modulators, three preclinical critical success factors were identified and met for zosuquidar: 1) it is a potent inhibitor of P-glycoprotein; 2) it is selective for P-glycoprotein; and 3) no pharmacokinetic interaction with co-administered chemotherapy is observed.

Zosuquidar is extremely potent in vitro (Kᵢ=59 nM) and is among the most active modulators of P-gp-associated resistance described to date. Zosuquidar has also demonstrated good in vivo activity in preclinical animal studies. In addition, the compound does not appear to be a substrate for P-gp efflux, resulting in a relatively long duration of reversal activity in resistant cells even after the modulator has been withdrawn.

Another significant attribute of zosuquidar as an MDR modulator is the minimal pharmacokinetic (PK) interactions with several oncolytics tested in preclinical models. Such minimal PK interaction permits normal doses of oncolytics to be administered and also a more straightforward interpretation of the clinical results.

The zosuquidar employed in formulations of preferred embodiments can be administered in the form of a pharmaceutically acceptable salt, e.g., the trihydrochloride salt. The terms “pharmaceutically acceptable salts” and “a pharmaceutically acceptable salt thereof” as used herein in regard to therapeutic agents are broad terms and are used in their ordinary sense, including, without limitation, to refer to salts prepared from pharmaceutically acceptable, non-toxic acids (e.g., as for zosuquidar) or bases (for other therapeutic agents capable of forming a salt with a base). Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of organic acids (e.g., benzenesulfonate, mesylate, fumarate, citrate), lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of zosuquidar or other therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl esters, protected derivatives, and chelates can also be suitable for use in compositions and methods of preferred embodiments. Also suitable for administration are selected therapeutic agents in hydrated form, selected enantiomeric forms of certain therapeutic agents, racemic mixtures of certain therapeutic agents, and the like.

Zosuquidar is generally administered in the form of the trihydrochloride salt. Conventional zosuquidar trihydrochloride formulations include those containing zosuquidar (50 mg as free base), glycine (15 mg), and mannitol (200 mg) dissolved in enough water for injection, to yield a free base concentration of 5 mg/mL. The formulation is filled into vials and lyophilized to give a vial containing 50 mg of free base. For such formulations, a 30 mL vial size is necessary to contain 50 mg of the zosuquidar formulation. For a typical >200 mg dose of zosuquidar, multiple 50 mg vials are needed to contain the formulation, greatly increasing manufacturing costs and reducing convenience for the end user (e.g., a pharmacist).

Modified Cyclodextrins

Cyclodextrins are cyclic oligomers of glucose; these compounds form inclusion complexes with any drug whose molecule can fit into the lipophile-seeking cavities of the cyclodextrin molecule. See U.S. Pat. No. 4,727,064 for a description of various cyclodextrin derivatives. Cyclodextrins of preferred embodiments can include α-, β-, and χ-cyclodextrins. The β-cyclodextrins include six glucopyranose units, the β-cyclodextrins include seven glucopyranose units, and the X-cyclodextrins include eight glucopyranose units. The β-cyclodextrins are generally preferred as having a suitable cavity size for zosuquidar. Cyclodextrin can be in any suitable form, including amorphous and crystalline forms, with the amorphous form generally preferred. Cyclodextrins suitable for use in the formulations of preferred embodiments include the hydroxypropyl, hydroxyethyl, glucosyl, maltosyl, and maltotrosyl derivatives of β-cyclodextrin, carboxyamidomethyl-β-cyclodextrin, carboxymethyl-β-cyclodextrin, and diethylamino-β-cyclodextrin.

Pharmaceutical complexes including various cyclodextrins and cyclodextrin derivatives are disclosed in the following United States patents: U.S. Pat. No. 4,024,223; U.S. Pat. No. 4,228,160; U.S. Pat. No. 4,232,009; U.S. Pat. No. 4,351,846; U.S. Pat. No. 4,352,793; U.S. Pat. No. 4,383,992; U.S. Pat. No. 4,407,795; U.S. Pat. No. 4,424,209; U.S. Pat. No. 4,425,336; U.S. Pat. No. 4,438,106; U.S. Pat. No. 4,474,881; U.S. Pat. No. 4,478,995; U.S. Pat. No. 4,479,944; U.S. Pat. No. 4,479,966; U.S. Pat. No. 4,497,803; U.S. Pat. No. 4,499,085; U.S. Pat. No. 4,524,068; U.S. Pat. No. 4,555,504; U.S. Pat. No. 4,565,807; U.S. Pat. No. 4,575,548; U.S. Pat. No. 4,598,070; U.S. Pat. No. 4,603,123; U.S. Pat. No. 4,608,366; U.S. Pat. No. 4,659,696; U.S. Pat. No. 4,623,641; U.S. Pat. No. 4,663,316; U.S. Pat. No. 4,675,395; U.S. Pat. No. 4,728,509; U.S. Pat. No. 4,728,510; and U.S. Pat. No. 4,751,095.

Chemically modified and substituted α, β-, and χ-cyclodextrins are generally preferred over unmodified α-, β-, and χ-cyclodextrins due to improved toxicity and solubility properties. The degree of substitution of the hydroxyl groups of the glucopyranose units of the cyclodextrin ring can affect solubility. In general, a higher average degree of −111-substitution of substituent groups in the cyclodextrin molecule yields a cyclodextrin of higher solubility.

Typically, only one guest molecule interacts with the cavity of the cyclodextrin to become entrapped. In order to form a complex with a cyclodextrin, a stable association is necessary. A variety of non-covalent forces, such as van der Waal forces, hydrophobic interaction, dipole moment and other forces are responsible for formation of a stable complex. In the case of some low molecular weight guest molecules, more than one guest molecule may fit into the cavity. In the case of some high molecular weight guest molecules, more than one molecule of cyclodextrin might bind to the guest molecule. Only a portion of the molecule must fit into the cavity to form a complex. As a result, a one-to-one molar ratio is not always achieved, especially with high or low molecular weight guest molecules. The guest molecule associates with the cyclodextrin so that the hydrophobic portion of the guest interacts with the hydrophobic cavity of the cyclodextrin. This interaction is an equilibrium reaction, with the direction of the equilibrium dependent upon the guest molecule. For some guest molecules, the complex is predominant while for other guest molecules, the free state might be preferred. In order to reduce the probability of free guest molecules self-associating to form an insoluble precipitate, excess cyclodextrin is frequently used to increase the probability of the guest molecule associating with the cavity of the cyclodextrin rather than associating with other guest molecules. For most modified cyclodextrins, a moderate excess of the cyclodextrin is generally desirable. However, in certain embodiments, a molar ratio of zosuquidar to the cyclodextrin approaching one-to-one may be preferred.

Sulfobutylcyclodextrin

Sulfobutyl-β-cyclodextrin is a particularly preferred modified cyclodextrin for solubilizing zosuquidar. This cyclodextrin is marketed by CyDex, Inc., (Lenexa, Kans.) under the trade name CAPTISOL®. CAPTISOL® cyclodextrins are polyanionic β-cyclodextrin derivatives with a sodium sulfonate salt separated from the lipophilic cavity by a butyl ether spacer group, or sulfobutylether (SBE). Sulfobutylcyclodextrin may provide a beneficial and protected environment for zosuquidar in its lipophilic cavity while its hydrophilic surface contributes good water solubility, improving both solubility and stability. Interaction of the zosuquidar with sulfobutylcyclodextrin may reduce decomposition by protecting the labile region from potential reactants in the aqueous environment. The inherent pharmacokinetics and pharmacodynamics of zosuquidar are unaffected by sulfobutylcyclodextrin. Upon administration, the zosuquidar—sulfobutylcyclodextrin complex rapidly disassociates, releasing zosuquidar.

Hydroxypropylcyclodextrin

Hydroxypropyl-β-cyclodextrin is also a preferred modified cyclodextrin for solubilizing zosuquidar. This cyclodextrin is marketed by RDI Division of Fitzgerald Industries Intl., (Concord, Mass.). Hydroxypropyl-β-cyclodextrin is produced from β-cyclodextrin by hydroxpropylation of the hydroxyl groups of the cyclodextrin. It is a partially substituted poly(hydroxpropyl) ether of beta cyclodextrin (BCD). The structure of a hydroxypropyl-β-cyclodextrin, wherein R═CH₂CH(OH)CH₃ or H, is as follows:

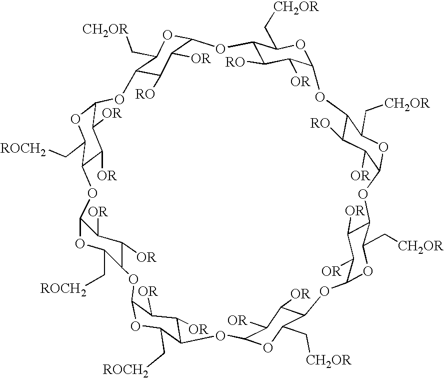

The basic closed circular structure of β-cyclodextrin is maintained in hydroxypropyl-β-cyclodextrin. The glycosidic oxygen forming the bond between the adjacent glucose monomers and the hydrogen atoms lining the cavity of the cyclodextrin impart an electron density and hydrophobic character to the cavity. Organic compounds, such as zosuquidar, interact with the walls of the cavity to form inclusion complexes. The hydroxyl groups and the hydroxypropyl groups are on the exterior of the molecule and interact with water to provide the increased aqueous solubility of the hydroxypropyl-β-cyclodextrin and the complexes made with the hydroxypropyl-β-cyclodextrin.

The hydroxypropyl groups are randomly substituted onto the hydroxyl groups of the β-cyclodextrin and the amount of substitution is reported as average degree of substitution or number of hydroxypropyl groups per β-cyclodextrin. In bulk hydroxypropyl-β-cyclodextrin, some molecules will have more substituents than the average number of substituents and some less. The result is a mixture of many molecules varying with respect to the number and location of substitutions around the ring of the β-cyclodextrin. Substitution can have an effect on the binding of guest molecules to the hydroxypropyl-β-cyclodextrin. At low degrees of substitution, binding is very similar to that of the unmodified β-cyclodextrin. Increasing substitution can lead to weakened binding due to steric hindrance. The effect is dependent upon the particular guest molecule, but it is possible to obtain increased binding due to an increase in surface area to which the guest molecule can bind. With most guest molecules, these differences in binding with degree of substitution are small. A preferred average degree of substitution of hydroxypropyl-β-cyclodextrin when employed in combination with zosuquidar is from about 4 or 5 to about 6, 7, or 8.

Hydroxypropyl-β-cyclodextrin is very soluble in water, with substitution of the hydroxyl groups of the β-cyclodextrin disrupting the network of hydrogen bonding around the rim of the β-cyclodextrin. As a result of disruption of the hydrogen-bonding network, the hydroxyl groups interact much more strongly with water, resulting in increased solubility compared to β-cyclodextrin. Hydroxypropyl-β-cyclodextrin is generally more soluble than unmodified β-cyclodextrin. For hydroxypropyl-β-cyclodextrin having a degree of substitution of 7.6, the solubility in aqueous solution is 360 g/100 ml. Hydroxypropyl-β-cyclodextrin is also soluble in aqueous ethanol (225 g/100 ml for a 95% ethanol solution). In preferred formulations, the solubility of the complex with zosuquidar is not generally exceeded. Complexes of zosuquidar and hydroxypropyl-β-cyclodextrins exhibit increased solubility and stability when compared to corresponding complexes of zosuquidar and unmodified β-cyclodextrins.

Strong acids, such as hydrochloric acids, can hydrolyze hydroxypropyl-β-cyclodextrin. The rate of hydrolysis is dependent upon the temperature and concentration of the acid. The higher the temperature or concentration of the acid, the more rapid is the rate of hydrolysis. Weak acids, such as organic acids, do not hydrolyze hydroxypropyl-β-cyclodextrin, and hydroxypropyl-β-cyclodextrin is stable in bases. Hydroxypropyl-β-cyclodextrin is not hydrolyzed by β-amylase or glucoamylase, but β-cyclodextrin can be hydrolyzed by some α-amylases. Hydroxypropyl-β-cyclodextrin generally exhibits good stability under physiological conditions when employed in formulations for intravenous use.

Zosuguidar—Sulfobutylcyclodextrin Formulations

While the preferred embodiments generally refer to zosuquidar—sulfobutylcyclodextrin formulations, it is understood that other suitable cyclodextrins, such as hydroxypropyl-β-cyclodextrins, can be used instead of sulfobutylcyclodextrin to solubilize zosuquidar. Alternatively, a mixture of two or more different cyclodextrins can be used.

Use of a sulfobutylcyclodextrin formulation (lyophilized) allows an 800 mg dose of zosuquidar to be contained in one (50 mL vial) or two vials (20 or 30 mL vial) versus three 100 mL vials for a zosuquidar formulation without cyclodextrin, resulting in greater manufacturing efficiency.

The relative amounts of zosuquidar and the cyclodextrin, e.g., sulfobutylcyclodextrin, can be adjusted, depending upon the particular formulation and the specific cyclodextrin employed. However, a molar ratio of zosuquidar to modified cyclodextrin of from about 1:1 or less to about 1:10 or more is generally preferred, preferably from about 1:5.0 or 1:5.5 to about 1:8.0, 1:8.5, 1:9.0, or 1:9.5, and more preferably from about 1:5.7, 1:5.8, 1:5.9, 1:6.0, 1:6.1, 1:6.2, 1:6.3, 1:6.4, 1:6.5, 1:6.6, 1:6.7 to about 1:6.8, 1:6.9, 1:7.0, 1:7.1, 1:7.2, 1:7.3, or 1.7:4.

The zosuquidar—modified cyclodextrin formulation can by supplied as a powder and reconstituted. Alternatively, it can be provided in the form of an aqueous liquid, which can optionally be freeze dried or lyophilized. In general, the zosuquidar—modified cyclodextrin formulations are prepared by dissolving the cyclodextrin in water and adding the zosuquidar to the aqueous modified cyclodextrin solution. Excipients, if any are desired may be added with or subsequent to adding the active compound. The resulting solution can be sterilized using any of the known methods appropriate to preserving the active compound. Alternatively, the components can be sterilized by any of the known methods appropriate to preserving zosuquidar prior to mixing in water and can be mixed using sterile equipment and techniques. The solution can be lyophilized in sterile containers and capped. Prior to use, the lyophilized composition of matter can be reconstituted using sterile water for injection, deionized sterilized water, 5% dextrose solution, or other appropriate diluent.

Contemplated routes of administration include topical, oral, subcutaneous, parenteral, intradermal, intramuscular, intraperitoneal, and intravenous. However, it is particularly preferred to administer the zosuquidar—modified cyclodextrin in intravenous form.

The intravenous forms containing zosuquidar—modified cyclodextrin are preferably isotonic with the blood or other body fluid of the patient. The isotonicity of the compositions can be attained using sodium tartrate, propylene glycol, sodium chloride, or other inorganic or organic solutes. Buffering agents can be employed, such as acetic acid and salts, citric acid and salts, boric acid and salts, and phosphoric acid and salts. Parenteral vehicles include, Ringer's dextrose, lactated Ringer's, or fixed oils. Intravenous vehicles can include fluid and nutrient replenishers, electrolyte replenishers (such as those based on Ringer's dextrose), and the like. A particularly preferred vehicle is dextrose solution, e.g., 5% dextrose. Various excipients can be employed, depending upon the route of administration and the preparation desired. Standard texts, such as “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively) include information regarding such excipients, which can include additional complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

A pharmaceutically acceptable preservative can be employed to increase the shelf life of the pharmaceutical compositions. Benzyl alcohol can be suitable, although a variety of preservatives including, for example, parabens, thimerosal, chlorobutanol, or benzalkonium chloride can also be employed. A suitable concentration of the preservative is typically from about 0.02% to about 2% based on the total weight of the composition, although larger or smaller amounts can be desirable depending upon the agent selected.

The zosuquidar—modified cyclodextrin complex can be provided to an administering physician or other health care professional in the form of a kit. The kit is a package which houses one or more containers which contain zosuquidar complexed with a modified cyclodextrin, such as sulfobutylcyclodextrin or hydroxypropyl-β-cyclodextrin, in a suitable form and instructions for reconstituting and/or administering the pharmaceutical composition to a subject. The kit can optionally also contain one or more additional therapeutic agents, e.g., mylotarg, daunorubicin, cytarabine, and/or other chemotherapeutic agents. The kit can optionally contain one or more diagnostic tools and instructions for use. For example, a kit containing a single composition comprising a complex of zosuquidar and sulfobutylcyclodextrin or hydroxypropyl-β-cyclodextrin in combination with one or more additional therapeutic agents can be provided, or separate pharmaceutical compositions containing a complex of zosuquidar—sulfobutylcyclodextrin and additional therapeutic agents can be provided. The kit can also contain separate doses of zosuquidar—sulfobutylcyclodextrin complex for serial or sequential administration. The kit can contain suitable delivery devices, e.g., syringes and the like, along with instructions for administrating the complex and any other therapeutic agent. The kit can optionally contain instructions for storage, reconstitution (if applicable), and administration of any or all therapeutic agents included. The kits can include a plurality of containers reflecting the number of administrations to be given to a subject. In a preferred embodiment, a kit for the treatment of a leukemia or solid tumor is provided. In a particularly preferred embodiment, a kit for the treatment of acute myelogenous leukemia is provided that includes a zosuquidar—sulfobutylcyclodextrin complex and mylotarg (for relapsed patients) or daunorubicin and cytarabine (for newly-diagnosed patients) and instructions for administering each. In another particularly preferred embodiment, a kit for the treatment of acute myelogenous leukemia is provided that includes a zosuquidar—sulfobutylcyclodextrin complex and one or more diagnostics or instructions for conducting one or more diagnostics for determining P-gp expression and/or efflux pump activity. The kit can also include instructions, an assay, or a diagnostic for determining if a patient has acute myelogenous leukemia. The kit can contain suitable delivery devices, e.g., syringes, inhalation devices, and the like, along with instructions for administrating zosuquidar and/or other therapeutic agent. The kit can optionally contain instructions for storage, reconstitution (if applicable, e.g. for a lyophilized form reconstituted for intravenous administration), and administration of any or all therapeutic agents included. The kits can include a plurality of containers reflecting the number of administrations to be given to a subject.

Contemplated amounts of solubilized zosuquidar for intravenous administration are from about 400 mg/day of zosuquidar or less to about 1,600 mg/day zosuquidar or more, preferably from about 500 or 600 mg/day to about 800, 900, 1000, 1100, 1200, 1300, 1400, or 1500 mg/day, and most preferably 700 mg/day. The duration of the injection of the zosuquidar—modified cyclodextrin complex can be adjusted depending upon various factors, and can comprise a single injection administered over the course of a few seconds or less to 1, 2, 3, 4, 5, 6, 7, 8, 9, 10, 11, 12, 13, 14, 15, 16, 17, 18, 19, 20, 21, 22, 23, 24, 26, 28, 30, 32, 34, 36, 40, 44, 48, 54, 60, 66, 72, 78, 84, 90, or 96 hours or more of continuous intravenous administration.

Chemotherapeutic Regimens Utilizing Zosuquidar—Sulfobutylcyclodextrin Formulations

The zosuquidar—sulfobutylcyclodextrin complex formulations of preferred formulations are useful therapeutic agents for treating multidrug resistance in patients treated for malignancies, solid tumors, and leukemias. The formulations are useful for treatment of cancers that express P-gp, e.g., many solid tumors, bladder cancer, pancreatic cancer, liver cancer, myeloma, carcinomas (e.g., breast cancer and ovarian cancer), sarcomas, and hematologic malignancies (e.g., acute myelogenous leukemia, acute lymphoblastic leukemia, chronic myeloid leukemia, plasma cell dyscrasias, lymphoma, myelodysplasia). The zosuquidar—sulfobutylcyclodextrin formulations are suitable for use in conjunction with suitable chemotherapeutic agents used to treat malignancies wherein multidrug resistance is of concern. However, the formulations are particularly suited for use in treating acute myelogenous leukemia. In preferred embodiments, relapsed patients are treated with mylotarg in combination with zosuquidar—sulfobutylcyclodextrin complex formulations. Newly-diagnosed patients can be treated with daunorubicin and cytarabine in combination with zosuquidar—sulfobutylcyclodextrin complex formulations. Other chemotherapeutic agents can also be used in combination with the zosuquidar—sulfobutylcyclodextrin complex formulations of preferred embodiments, e.g., anthracyclines (e.g., doxorubicin, daunorubicin, epirubicin, idarubicin, mitoxantrone), vincas (e.g., vincristine, vinblastine, vinorelbine, vindesine), Topoisomerase-II (e.g., etoposide, teniposide), taxanes (e.g., paclitaxel, docetaxel), and others (e.g., Gleevec, Mylotarg, dactinomycin, mithramycin).

Chemotherapeutic Regimens Utilizing Zosuquidar and Mylotarg

In preferred embodiments, a P-gp expression or efflux pump activity diagnostic is conducted to provide information in treating AML patients or patients with metastatic breast cancer with a zosuquidar—cyclodextrin complex (e.g., zosuquidar—sulfobutylcyclodextrin or zosuquidar—hydroxypropyl cyclodextrin) in combination with Mylotarg. If the results of the P-gp expression or efflux pump activity diagnostic indicates positive P-gp expression or efflux pump activity, then treatment with a zosuquidar—cyclodextrin complex in combination with Mylotarg is initiated. If the results of the P-gp expression or efflux pump activity diagnostic indicate negative P-gp expression or efflux pump activity, then zosuquidar is expected not to yield an improvement in clinical outcome and another treatment option not involving administration of a P-gp efflux inhibitor is selected. In relapsed AML patients, it is generally considered acceptable clinical practice to wait for P-gp expression or efflux pump activity test results before initiating a treatment. However, in certain embodiments it can be desirable to initiate treatment before receiving test results, and then reevaluate the desirability of continuing treatment, depending upon the test results. Most preferably, P-gp expression or efflux pump activity of a sample both in the presence and absence of the P-gp efflux inhibitor is compared, whereby the P-gp efflux that is inhibitable by the P-gp efflux inhibitor can be determined. However, in certain embodiments wherein P-gp expression or function status correlates with expectation of clinical success, it can be useful to determine P-gp expression or efflux pump activity at any point in time.

Mylotarg was approved in May 2000 for relapsed CD33-positive AML patients over the age of 60. Mylotarg from Wyeth and Celltech is based on antibody-targeted chemotherapy. Mylotarg's highly specific antibody recognizes a cell-surface molecule, CD33, which is abundant on AML cells (>90%) but absent from normal blood stem cells, the seeds from which normal blood and immune cells originate. The antibody is linked to calicheamicin, a potent chemotherapy agent. The antibody selectively targets leukemic blast cells and delivers calicheamicin to them. The chemical structure of Mylotarg is provided below.

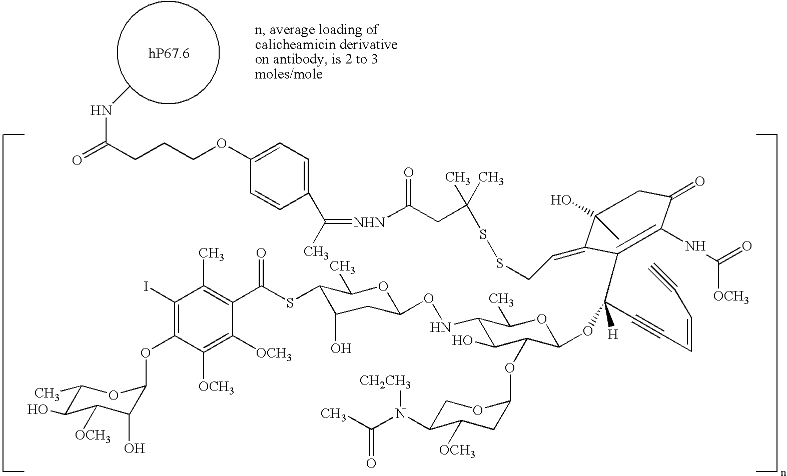

There is a growing body of evidence to suggest that the calicheamicin component of Mylotarg is also an MDR substrate and subject to the P-gp efflux pump. In several studies, the cytotoxic effect of Mylotarg has been shown to be inversely correlated with the amount of P-gp present. Two MDR modulators, valspodar and the quinolone derivative MS-209, have both been shown to reverse the resistance to Mylotarg in P-gp expressing CD33(+) leukemia cells and clinical studies are underway in combination with cyclosporine.

The combination of zosuquidar, a highly specific and safe P-gp efflux inhibitor, complexed with cyclodextrin, in combination with Mylotarg or another calicheamicin-antibody conjugate is effective for treatment of relapsed AML. The effective dose of the zosuquidar—cyclodextrin complex and the timing of administration of zosuquidar and Mylotarg are critical to achieving improved complete remission rates and enhanced leukemia free and overall survival rates in the relapsed AML patient population. While the methods and formulations of preferred embodiments are especially preferred for treatment of relapsed AML patients, the methods and formulations can be adapted to other drugs and indications. For example, P-gp efflux inhibitors other than Mylotarg can be administered according to the disclosed dosing regimens, or slightly modified dosing regimens. Likewise, the formulations and dosing regimens employing a zosuquidar—cyclodextrin complex and Mylotarg can be employed in treating AML patients other than relapsed AML patients, or for other types of leukemia or other cancers that express P-gp, e.g., many solid tumors, lymphomas, bladder cancer, pancreatic cancer, ovarian cancer, liver cancer, myeloma, lymphocytic leukemia, breast cancer, and sarcoma.

The duration of the injection of a zosuquidar—cyclodextrin complex and/or Mylotarg can be adjusted depending upon various factors, and can comprise a single injection administered over the course of a few seconds or less to 1, 2, 3, 4, 5, 6, 7, 8, 9, 10, 11, 12, 13, 14, 15, 16, 17, 18, 19, 20, 21, 22, 23, 24, 26, 28, 30, 32, 34, 36, 40, 44, 48, 54, 60, 66, 72, 78, 84, 90, or 96 hours or more of continuous intravenous administration.

A zosuquidar—cyclodextrin complex and a therapeutic agent that is a substrate for P-gp efflux can be administered to patients suffering from AML prior to confirmation of P-gp expression or function, or to AML patients other than relapse AML patients. However, such therapy is preferably administered to relapsed AML patients. The administration route, amount administered, and frequency of administration can vary depending on the age of the patient, status as relapsed or newly diagnosed AML patient, and severity of the condition.

Contemplated amounts of Mylotarg for intravenous administration to treat relapsed AML are from about 10 mg/day or less to about 1000 mg/day or more administered on one, two, or more separate days. The dosage is preferably administered intravenously at a rate of about 1 mg/m² or less to about 10 mg/m² or more continuously over the course of about 2, 3, or 4 hours to about 5, 6, 7, 8, 9, 10, 11, 12, 13, 14, 15, 16, 17, 18, 19, 20, 21, 22, 23, or 24 hours, more preferably over the course of about 2 hours to about 6 hours; however, administration at a rate of 5 mg/m², 7 mg/m², or 9 mg/m² over about 2 hours is particularly preferred. Preferably, doses of Mylotarg are administered on Day 1 and Day 15 of the treatment regimen. However, in certain embodiments, the second dose can be administered on Day 5, 6, 7, 8, 9, 10, 11, 12, 13, 14, 16, 17, 18, 19, 20, 21, or 22, or another day of the treatment regimen. Other dosing regimens include administering three doses total over a week.

Contemplated amounts of zosuquidar (in the form of a cyclodextrin complex) for intravenous administration to treat relapsed AML are from about 400 mg/day or less to about 1,600 mg/day or more, preferably from about 500, 600, or 700 mg/day to about 900, 1000, 1100, 1200, 1300, 1400, or 1500 mg/day, and most preferably from about 500 mg/day to about 800 mg/day. It is generally preferred to start the infusion of the zosuquidar—cyclodextrin complex from about 2 hours or less to about 6 hours or more prior to the administration of Mylotarg. In the course of a treatment regimen, the zosuquidar—cyclodextrin complex is preferably administered on two, three, or four separate days. The dosage is preferably administered intravenously continuously over the course of about 6 to 90 hours, more preferably over the course of 12, 18, 24, 30, 36, or 42 hours to about 54, 60, 66, 72, 78, or 84 hours, most preferably over about 24 hours, 48 hours, or 72 hours, depending upon the treatment regimen. Preferably the zosuquidar—cyclodextrin complex is administered on Day 1 of the treatment regimen. In certain embodiments, additional zosuquidar—cyclodextrin complex is administered on Day 2, on Days 2 and 3, or on Days 2, 15, and 16. However, in certain embodiments, one, two, or three or more additional doses can be administered on other days of the treatment regimen.

Table 1 provides various dosing regimes that can be used in treating relapsed AML.

TABLE 1

| Dose | | Zosuquidar |
|---|---|---|
| Level | Mylotarg | (complexed with cyclodextrin) |
| −1* | 5 mg/m² IV over 4 hr | 800 mg/day continuous IV over 24 hr |
| | Day 1 and 15 | Day 1 and 15 |
| 1 | 5 mg/m² IV over 4 hr | 800 mg/day continuous IV over 48 hr |
| | Day 1 and 15 | Day 1&2 and 15&16 |
| 2 | 7 mg/m² IV over 4 hr | 800 mg/day continuous IV over 48 hr |
| | Day 1 and 15 | Day 1&2 and 15&16 |
| 3 | 9 mg/m² IV over 4 hr | 800 mg/day continuous IV over 48 hr |
| | Day 1 and 15 | Day 1&2 and 15&16 |
| 4 | 9 mg/m² IV over 4 hr | 800 mg/day continuous IV over 72 hr |
| | Day 1 and 15 | Day 1-3 and 15-17 |

*Only if level 1 has a dose limiting toxicity (DLT).

Tables 2 and 3 provide alternative dosing regimes that can be used in relapsed AML.

TABLE 2

| Dose | | Zosuquidar |
|---|---|---|
| Level | Mylotarg | (complexed with cyclodextrin) |
| −1* | 5 mg/m² IV over | 500-700 mg/day continuous IV over 24 hr |
| | 6-24 hr Day 1 and 15 | Day 1 and 15 |
| 1 | 5 mg/m² IV over | 500-700 mg/day continuous IV over 48 hr |
| | 6-24 hr Day 1 and 15 | Day 1&2 and 15&16 |
| 2 | 7 mg/m² IV over | 500-700 mg/day continuous IV over 48 hr |
| | 6-24 hr Day 1 and 15 | Day 1&2 and 15&16 |
| 3 | 9 mg/m² IV over | 500-700 mg/day continuous IV over 48 hr |
| | 6-24 hr Day 1 and 15 | Day 1&2 and 15&16 |
| 4 | 9 mg/m² IV over | 500-700 mg/day continuous IV over 72 hr |
| | 6-24 hr Day 1 and 15 | Day 1-3 and 15-17 |

*Only if level 1 has a dose limiting toxicity (DLT).

A clinical study was conducted to determine the efficacy of Mylotarg in the treatment of relapsed AML. It was determined that the rate of complete remission (CR+CRp) for P-gp negative patients treated with Mylotarg was 64% (N=36). In contrast, the rate of complete remission for P-gp positive patients was only 9% (N=22). This indicates that P-gp efflux plays an important role in survival rates for relapsed AML, and further indicates that inhibition of P-gp efflux, e.g., by also administering zosuquidar or another P-gp efflux inhibitor, has the potential to significantly improve response rates in P-gp positive patients. The diagnostic and assay methods described herein are therefore useful in treating relapsed AML. Likewise, a diagnostic or assay to determine P-gp expression or function or efflux pump activity can be useful in devising treatment regimens for other cancers, such as metastatic breast cancer, that also exhibit P-gp expression.

Chemotherapeutic Regimens Utilizing Zosuquidar, Daunorubicin, and Cytarabine

In preferred embodiments, a P-gp expression or efflux pump activity diagnostic is conducted to provide information in treating newly diagnosed AML patients with a zosuquidar—cyclodextrin complex (e.g., zosuquidar—sulfobutylcyclodextrin or zosuquidar—hydroxypropyl cyclodextrin) in combination with daunorubicin and cytarabine. In newly diagnosed AML patients, it is generally not considered acceptable clinical practice to wait for P-gp expression or efflux pump activity test results before initiating a treatment. Accordingly, treatment is initiated immediately after diagnosis. When test results become available, the desirability of continuing treatment can be evaluated, depending upon the test results. Typically, when the results of the P-gp expression or efflux pump activity diagnostic indicate negative P-gp expression, then treatment with a P-gp efflux inhibitor is discontinued because administration of the drug is not expected to contribute to an improved clinical outcome. Preferably, P-gp expression or function or efflux pump activity is determined both in the presence and the absence the P-gp efflux inhibitor to determine the P-gp expression that is inhibitable by the P-gp efflux inhibitor.

Daunorubicin is an antibiotic chemotherapy treatment that is widely used to treat acute myeloid leukemia and acute lymphocytic leukemia. It is produced by the bacteria Streptomyces coeruleorubidis and was approved by the FDA as a first line therapy treatment for leukemia in 1998. Daunorubicin is typically administered intravenously. It is marketed under the brand names Cerubidine, DaunoXome, and Liposomal daunorubicin. Daunorubicin has the following structure:

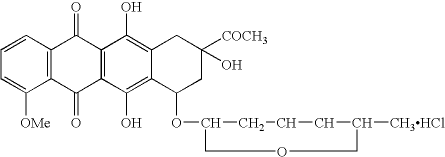

Cytarabine is a deoxycytidine analogue, cytosine arabinoside (ara-C), which is metabolically activated to the triphosphate nucleotide (ara-CTP), which acts as a competitive inhibitor of DNA polymerase and produces S phase-specific cytotoxicity. It is used as an antineoplastic, generally as part of a combination chemotherapy regimen, in the treatment of acute lymphocytic and acute myelogenous leukemia, the blast phase of chronic myelogenous leukemia, erythroleukemia, and non-Hodgkin's lymphoma. It is typically administered intravenously and subcutaneously, and for the prophylaxis and treatment of meningeal leukemia, administered intrathecally. Cytarabine has the following structure:

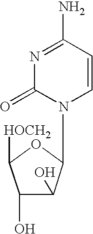

The combination of a zosuquidar—cyclodextrin complex, the antibiotic chemotherapeutic daunorubicin, and the antineoplastic cytarabine, is effective for treatment of newly diagnosed AML. The effective dose of the zosuquidar—cyclodextrin complex and the timing of administration of the zosuquidar—cyclodextrin complex, daunorubicin, and cytarabine are critical to achieving improved complete remission rates and enhanced leukemia free survival rates in the newly diagnosed AML patient population. While the methods and formulations of preferred embodiments are especially preferred for treatment of newly diagnosed AML patients, the methods and formulations can be adapted to other drugs and indications. For example, chemotherapeutics other than daunorubicin and cytarabine can be administered according to the disclosed dosing regimens, or slightly modified dosing regimens. Likewise, the formulations and dosing regimens employing a zosuquidar—cyclodextrin complex, daunorubicin, and cytarabine can be employed in treating AML patients other than newly diagnosed AML patients, or for treating other types of leukemia or other cancers that exhibit P-gp expression.

Zosuquidar—cyclodextrin complex, daunorubicin, and cytarabine can be formulated as described above for zosuquidar—cyclodextrin complex and Mylotarg, and can be included in kits, also as described above.

The zosuquidar—cyclodextrin complex, daunorubicin, and/or cytarabine can be administered to patients suffering from AML prior to confirmation of the P-gp expression or function, or to AML patients other than newly diagnosed AML patients (e.g., relapsed AML patients). However, therapy is preferably administered to newly diagnosed AML patients. The administration route, amount administered, and frequency of administration can vary depending on the age of the patient, status as relapsed or newly diagnosed AML patient, and severity of the condition

Contemplated amounts of zosuquidar (in the form of a cyclodextrin complex) for intravenous administration to treat newly diagnosed AML are from about 400 mg/day or less to about 1,600 mg/day or more, preferably from about 500, 600, or 700 mg/day to about 900, 1000, 1100, 1200, 1300, 1400, or 1500 mg/day, and most preferably 700 mg/day. In the course of a treatment regimen, the zosuquidar—cyclodextrin complex is preferably administered on two, three, or four separate days. The dosage is preferably administered in intravenously continuously over the course of about 6 to about 90 hours, more preferably over the course of about 12, 18, 24, 30, 36, or 42 hours to about 54, 60, 66, 72, 78, or 84 hours, most preferably over about 24 hours, 48 hours, or 72 hours, depending upon the treatment regimen. Preferably the zosuquidar—cyclodextrin complex is administered on Day 1 of the treatment regimen. In certain embodiments, additional zosuquidar—cyclodextrin complex is administered on Day 2, on Days 2 and 3, or on Days 2, 15, and 16. However, in certain embodiments, one, two, or three or more additional doses can be administered on other days of the treatment regimen.

Contemplated amounts of daunorubicin for intravenous administration to treat newly diagnosed AML are from about 10 mg/m²/day or less to about 100 mg/m²/day or more administered at initiation of zosuquidar—cyclodextrin complex infusion or up to about 1, 2, 3, 4, 5, or 6 or more hours after initiation of zosuquidar—cyclodextrin complex infusion. The dosage is preferably administered intravenously at a rate of about 25 mg/m²/day or less to about 90 mg/m²/day or more, preferably about 30, 35, or 40 mg/m²/day or less to about 50, 55, 60, 65, 70, 75, 80, or 85 mg/m²/day, and most preferably about 45 mg/m²/day continuously over the course of about 2 or 2.5 days to about 3.5 or 4 days, preferably about 3 days.

Contemplated amounts of cytarabine for intravenous administration to treat newly diagnosed AML patients are from about 10 mg/day or less to about 3,000 mg/day or more administered at initiation of zosuquidar—cyclodextrin complex infusion or after initiation of zosuquidar—cyclodextrin complex infusion. The dosage is preferably administered intravenously at a rate of about 50 mg/m²/day or less to about 200 mg/m²/day or more, preferably 60, 70, 80, or 90 mg/m²/day or less to about 110, 120, 130, 140, 150, 160, 170, 180, or 190 mg/m²/day, and most preferably about 100 mg/m²/day continuously over the course of about 1, 2, 3, 4, 5, or 6 days up to about 8, 9, or 10 days or more, preferably over about 7 days.

A particularly preferred dosing regimen for newly diagnosed AML includes continuous intravenous administration of 550 mg of zosuquidar (as a cyclodextrin complex) over 6 hours (3 days), continuous intravenous administration of cytarabine at a rate of 100 mg/m²/day (7 days), and intravenous administration of daunorubicin at a dose of 45 mg/m²/day (3 days), wherein infusion of daunorubicin is started 1 hour after initiation of zosuquidar infusion. Another particularly preferred dosing regimen includes continuous intravenous administration (preferably about 1 to 24 hours in duration, more preferably about 6 to 24 hours in duration, most preferably about 24 hours in duration) of 500 to 700 mg/day of zosuquidar (3 days), continuous intravenous administration of cytarabine at a rate of 100 mg/m²/day (7 days), and intravenous administration of daunorubicin at a dose of 45 mg/m²/day (3 days), wherein infusion of daunorubicin is started 1 to 4 hours after initiation of zosuquidar—cyclodextrin complex infusion. While in the above described embodiments infusion of daunorubicin is started after a specified time period has lapsed after initiation of zosuquidar—cyclodextrin complex infusion, in other embodiments other start times can be preferred, e.g., immediately after or during initiation of zosuquidar—cyclodextrin complex infusion up to about 1, 2, 3, 4, 5, 6, 7, 8, 9, 10, 11, 12, or more hours after initiation of zosuguidar—cyclodextrin complex infusion.

Experiments

Dissolution testing was conducted to determine fill volumes required to achieve an 800 mg dose of zosuquidar at various concentrations of sulfobutylcyclodextrin CAPTISOL®, β-cyclodextrin derivative sodium salt, CyDex, Inc., Lenexa, Kans.). CAPTISOL® is a polyanionic β-cyclodextrin derivative with a sodium sulfonate salt separated from the lipophilic cavity by a butyl ether spacer group, or sulfobutylether. Upon intravenous administration, CAPTISOL® exhibits limited plasma protein binding and distributes to intracellular fluid. IV doses of ¹⁴C-labeled CAPTISOL administered to rats, mice, dogs, rabbits and humans were rapidly and completely cleared intact from the circulation. Excretion is primarily in urine, with clearance approximating the glomerular filtration rate.

As demonstrated in the data of Table 1, acceptable fill volumes for 800 mg zosuquidar can be achieved for solutions containing from about 8 mg/mL to about 50 mg/mL zosuquidar, and from about 5 wt. % (based on solvent, i.e., water, weight) to about 30 wt. % (based on solvent, i.e., water, weight) of sulfobutylcyclodextrin. Larger or smaller amounts of zosuquidar can be filled into vials by varying fill volume.

TABLE 1
Dissolution Summary - 800 mg Zosuquidar

| | | Sulfobutyl | | | | | *Fill | *Fill |
|---|---|---|---|---|---|---|---|---|
| | Zosuquidar | Cyclodextrin | Tween | Glycine | | | Volume | Volume |
| Sample | mg/mL | (solvent wt.) | 80 | mg/mL | pH | Dissolution | 1 Vial | 2 Vials |
| 1 | 50.00 | 30% | | | 1.33 | 6 min 5 sec | 16.0 mL | 8.0 mL |
| 2 | 41.67 | 25% | | | 1.37 | 5 min 5 sec | 19.2 mL | 9.6 mL |
| 3 | 33.33 | 20% | | | 1.47 | 1 min 50 sec | 24.0 mL | 12.0 mL |
| 4 | 33.33 | 20% | | 5 | 1.49 | 0 min 55 sec | 24.0 mL | 12.0 mL |
| 5 | 33.33 | 20% | | 20 | 2.19 | 1 min 5 sec | 24.0 mL | 12.0 mL |
| 6 | 33.33 | 20% | 0.1% | | 3.09 | 1 min 55 sec | 24.0 mL | 12.0 mL |
| 7 | 33.33 | 20% | 0.25% | | 1.56 | 2 min 10 sec | 24.0 mL | 12.0 mL |
| 8 | 25.00 | 15% | | | 1.54 | 0 min 55 sec | 32.0 mL | 16.0 mL |
| 9 | 16.67 | 10% | | | 1.69 | <30 sec | 48.0 mL | 24.0 mL |
| 10 | 8.33 | 5% | | | 1.94 | <30 sec | 96.0 mL | 48.0 mL |

*Based on an 800 mg dose

Data were also obtained demonstrating the feasibility of achieving acceptable fill volume for 900 mg zosuquidar (Table 2).

TABLE 2
Dissolution Summary - 900 mg Zosuquidar

| | | | | | | **Fill |
|---|---|---|---|---|---|---|
| | Zosuquidar | Sulfobutyl | | | **Fill Volume | Volume |
| Sample | mg/mL | cyclodextrin | pH | Dissolution | 1 Vial | 2 Vials |
| 1a | 65.2 mg/mL | 30% | 1.19 | 7.5 minutes | 13.8 mL | 6.9 mL |
| 4a | 43.0 mg/mL | 20% | 1.30 | 2.5 minutes | 20.9 mL | 10.5 mL |

The data suggest that the solubility of the formulations is pH sensitive. When formulated at 50 mg/mL zosuquidar and 30% sulfobutylcyclodextrin, the normal pH is approximately 1.3. Titration experiments showed that when the pH of this solution was increased to around 3.5, active ingredient precipitated out of solution. Based on this observation, material was formulated and the pH adjusted to 3.0 with NaOH. The samples were then freeze-dried. The active ingredients exhibited satisfactory solubility in solution at this pH. However, when diluted into IV administration fluids (normal saline or 5% dextrose (900 mg-500 mL)) there was a significant decrease in solubility. The normal saline solution became hazy immediately. The 5% dextrose solution became turbid over the course of 1 hour. The pH of both solutions was determined and found to be 3.97 and 4.05 for saline and dextrose, respectively. Phosphoric acid was added to each formulation until the pH was measured at less than or equal to 2, and the haziness disappeared. The data suggests that the common ion effect plays a small role in precipitation; however, pH appears to be a major force.

Solubility studies were conducted using zosuquidar and various co-solvents and complexing agents. Solutions, as described in Table 3, were formulated and 3 mL aliquots were placed in 6 mL scintillation vials. An excess of zosuquidar was added to each solution and the vials were capped. Samples were placed on a Burrell Model 75 shaker and shaken on high speed. The samples were watched over the course of several hours and additional zosuquidar was added where needed. The samples were shaken for approximately 20 hours. The samples were removed from the shaker and visually observed. Table 4 provides the visual data results.

TABLE 3
Formulations for Solubility Testing

| Sample # | Formulation |
|---|---|
| 1 | Purified Water |
| 2 | 5% Ethanol |
| 3 | 10% Ethanol |
| 4 | 15% Ethanol |
| 5 | 20% Propylene Glycol 5% Ethanol |
| 6 | 20% Sulfobutylcyclodextrin 5% Ethanol |
| 7 | 2.5% Sulfobutylcyclodextrin |
| 8 | 5% Sulfobutylcyclodextrin |
| 9 | 10% Sulfobutylcyclodextrin |
| 10 | 20% Sulfobutylcyclodextrin |
| 11 | 40% Sulfobutylcyclodextrin |
| 12 | 20% Sulfobutylcyclodextrin pH 5 |
| 13 | 20% Sulfobutylcyclodextrin pH 7 |
| 14 | 20% Sulfobutylcyclodextrin pH 9 |

TABLE 4
Formulations for Solubility Testing

| Sample # | Visual Results |
|---|---|
| 1* | Thin, milky yellow suspension |
| 2* | Very viscous (gelled), milky yellow suspension. Some aggregated solids were present |
| 3* | Very viscous (gelled), milky yellow suspension. Some aggregated solids were present |
| 4 | Thin, milky yellow suspension |
| 5* | Thin, milky yellow, pearlescent suspension |
| 6 | Thin, clear, yellow solution containing undissolved, aggregated solids |
| 7 | Thin, clear, yellow solution containing undissolved, aggregated solids |
| 8 | Thin, clear, yellow solution containing undissolved, aggregated solids |
| 9 | Thin, clear, yellow solution containing undissolved, aggregated solids |
| 10 | Thin, clear, yellow solution containing undissolved, aggregated solids |
| 11 | Slightly viscous, clear, yellow solution containing undissolved, aggregated solids |
| 12* | Thin, milky yellow suspension |
| 13 | Thin, clear, yellow solution containing undissolved, aggregated solids |
| 14 | Thin, clear, yellow solution containing undissolved, aggregated solids |

*Samples 1, 2, 3, 5, and 12 could not be filtered - undissolved solids finely suspended

The samples were filtered through 0.45 μm syringe filters to remove the undissolved solids. Samples 1, 2, 3, 5, and 12 could not be filtered because the undissolved solids were so finely suspended that the filter was easily blocked. These samples were centrifuged at 4500 rpm in an attempt to separate the solids; however, only samples 5 and 12 could be separated. Sample 11, although clear, was too viscous to pass through the 0.45 μm membrane and so was instead filtered using a 5 μm membrane. The potency of each sample (if able to be separated) was determined using an HPLC potency assay. The results (reported as the free base) are listed in Table 5.

TABLE 5
Formulations for Solubility Testing

| Sample # | Potency (Free base), mg/mL |
|---|---|
| 15% Ethanol | 13.7 |
| 20% Propylene Glycol 5% Ethanol | 21.8 |
| 20% Sulfobutylcyclodextrin 5% Ethanol | 30.9 |
| 2.5% Sulfobutylcyclodextrin | 5.5 |
| 5% Sulfobutylcyclodextrin | 3.9 |
| 10% Sulfobutylcyclodextrin | 9.7 |
| 20% Sulfobutylcyclodextrin (pH 6.4) | 32.0 |
| 40% Sulfobutylcyclodextrin | 87.1 |
| 20% Sulfobutylcyclodextrin pH 5 | 34.2 |
| 20% Sulfobutylcyclodextrin pH 7 | 29.5 |
| 20% Sulfobutylcyclodextrin pH 9 | 30.6 |

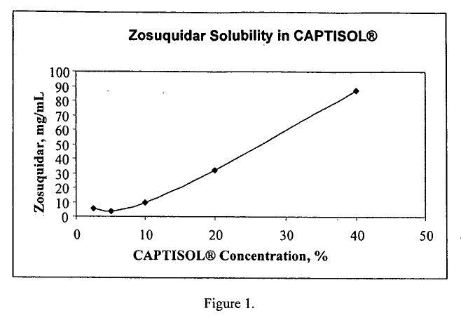

The data demonstrate that the solubility of zosuquidar is significantly increased when CAPTISOL® (β-cyclodextrin derivative sodium salt) is incorporated into the formulation. The graph in FIG. 1 illustrates the increase of zosuquidar solution concentration as a function of sulfobutylcyclodextrin concentration.

A study was conducted to determine the optimum ratio of zosuquidar to CAPTISOL®. Additionally, the use of glycine and Polysorbate 80 additions were investigated as a means of decreasing dissolution time. The different formulations tested are listed in Table 6. After formulation, the clear solutions were separated from the hazy solutions and 2 mL aliquots were placed into 5 mL×13 mm vials and were freeze-dried. After lyophilization, the vials were removed and a visual description was recorded. All cakes were yellow, slightly shrunken, and showed no signs of collapse. Samples were reconstituted with 2 mL of purified water, the dissolution time was recorded, and a visual description of the reconstituted solution was recorded. Solutions containing a haze or insoluble material were shaken for at least five minutes before being listed as N/A.

TABLE 6

| | Zosuquidar | % | % Tween | Glycine | Solution | Dissolution | Reconstituted |
|---|---|---|---|---|---|---|---|
| Sample | mg/mL | CAPTISOL ® | 80 | mg/mL | Description | Time | Description |
| 1 | 10.8 | 5 | 0 | 0 | very sl. haze | n/a | n/a |
| 2 | 10.8 | 5 | 0.25 | | very sl. haze | n/a | n/a |
| 3 | 10.8 | 5 | | 5 | sl. haze | n/a | n/a |
| 4 | 10.8 | 5 | | 10 | hazy | n/a | n/a |
| 5 | 10.8 | 5 | | 20 | hazy | n/a | n/a |
| 6 | 21.5 | 10 | 0 | 0 | Clear | 45 secs. | Insoluble matter |
| | | | | | | | in vial |
| 7 | 21.5 | 10 | 0.25 | | Clear | 35 secs | Small bead of |
| | | | | | | | insoluble matter |
| 8 | 21.5 | 10 | | 5 | hazy | n/a | n/a |
| 9 | 21.5 | 10 | | 10 | sl. haze | n/a | n/a |
| 10 | 21.5 | 10 | | 20 | sl. haze | n/a | n/a |
| 11 | 32.3 | 15 | 0 | 0 | Clear | 3.3 min. | Some Insoluble |
| | | | | | | | matter in vial |
| 12 | 32.3 | 15 | 0.25 | | Clear | 3.2 min. | Small bead of |
| | | | | | | | insoluble matter |
| 13 | 32.3 | 15 | | 5 | Clear | 2.7 min. | Some Insoluble |
| | | | | | | | matter in vial |
| 14 | 32.3 | 15 | | 10 | very sl. haze | n/a | n/a |
| 15 | 32.3 | 15 | | 20 | sl. haze | n/a | n/a |
| 16 | 43.0 | 20 | 0 | 0 | Clear | 3.2 min. | Some Insoluble |
| | | | | | | | matter in vial |
| 17 | 43.0 | 20 | 0.25 | | Clear | 4.3 min | Some Insoluble |
| | | | | | | | matter in vial |
| 18 | 43.0 | 20 | | 5 | Clear | 3.3 min. | No insoluble |
| | | | | | | | matter observed |
| 19 | 43.0 | 20 | | 10 | Clear | 2 min | Some Insoluble |
| | | | | | | | matter in vial |
| 20 | 43.0 | 20 | | 20 | very sl. haze | n/a | n/a |

Based on the results from Table 6, while some of the samples with higher concentrations do go completely into solution when formulating, most of them do not reconstitute in an acceptable amount of time and or go completely back into solution. Neither Tween 80 nor glycine had a significant impact on the dissolution time or the reconstitution solubility. The only sample that did reconstitute to a complete solution was sample 18; however, the reconstitution time was relatively high considering this was a 2 mL fill and reconstitution times would likely increase as the sample volume increased.

An additional study was conducted to further investigate the ratio of zosuquidar to CAPTISOL® and the effects on dissolution time and completeness of solution after reconstitution. Samples were formulated 2 mL aliquots were placed into 5 mL×13 mm vials and were freeze-dried using a conservative cycle. After drying, all of the samples were inspected, and all vials contained a yellow, slightly shrunken plug with no signs of collapse. Samples were reconstituted with 2 mL of purified water and the reconstitution times were recorded. Upon inspection of the reconstituted solutions, all samples visually formed a complete solution with no undissolved solids stuck to the sides of the vial or floating free in solution. Table 7 lists the different samples tested and the results. The theoretical fill volumes of 1 vial per dose and 2 vials per dose based on an 800 mg dose are included in this table.

TABLE 7

| | | | | | | | *Fill | *Fill |
|---|---|---|---|---|---|---|---|---|
| | Zosuquidar | | Tween | Glycine, | | | Volume | Volume |
| Sample | mg/mL | CAPTISOL ® | 80% | mg/mL | pH | Dissolution | 1 Vial | 2 Vials |
| 1 | 50.00 | 30% | | | 1.33 | 6 min 5 sec | 16.0 mL | 8.0 mL |
| 2 | 41.67 | 25% | | | 1.37 | 5 min 5 sec | 19.2 mL | 9.6 mL |
| 3 | 33.33 | 20% | | | 1.47 | 1 min 50 sec | 24.0 mL | 12.0 mL |
| 4 | 33.33 | 20% | | 5 | 1.49 | 0 min 55 sec | 24.0 mL | 12.0 mL |
| 5 | 33.33 | 20% | | 20 | 2.19 | 1 min 5 sec | 24.0 mL | 12.0 mL |
| 6 | 33.33 | 20% | 0.1% | | 3.09 | 1 min 55 sec | 24.0 mL | 12.0 mL |
| 7 | 33.33 | 20% | 0.25% | | 1.56 | 2 min 10 sec | 24.0 mL | 12.0 mL |
| 8 | 25.00 | 15% | | | 1.54 | 0 min 55 sec | 32.0 mL | 16.0 mL |
| ** | 22.5 | 15% | | | 1.6 | 1 min 30 sec | — | 12.5 mL |
| 9 | 16.67 | 10% | | | 1.69 | <30 sec | 48.0 mL | 24.0 mL |
| 10 | 8.33 | 5% | | | 1.94 | <30 sec | 96.0 mL | 48.0 mL |

*Based on an 800 mg dose
** Preferred formulation based on a 550 mg dose

The results shown in Table 7 show that based on dissolution time, Tween 80 does not improve the reconstitution properties of the formulation. In contrast, Tween 80 appears to slow down the dissolution time. Adding glycine to the formulation did appear to offer some benefit in reducing the dissolution time.

Samples were formulated containing 20% CAPTISOL®, 33.3 mg/mL of zosuquidar, and different amounts of glycine. 2 mL aliquots were placed into 5 mL×13 mm vials and were freeze-dried using a conservative cycle. Half of the samples were held aside and freeze-dried using a conservative cycle with an annealing step (hold at −15° C. for 2 hours prior to re-cooling back to −45° C. and freeze-drying). All of the vials contained yellow cakes, which were slightly shrunken, and no signs of collapse were observed. Samples were reconstituted with 2 mL of purified water, and the dissolution time and the description of the solution was recorded. Table 8 contains the samples tested and the results.

TABLE 8

| Sample | Lyo Cycle | Glycine, mg/mL | Dissolution Time | Description |
|---|---|---|---|---|
| 1 | Normal | 0 | 1 min. 51 secs | No insoluble matter present |
| 2 | Normal | 1 | 1 min. 53 secs | No insoluble matter present |
| 3 | Annealed | 1 | 2 min. 28 secs | No insoluble matter present |
| 4 | Normal | 2 | 1 min. 46 secs | No insoluble matter present |
| 5 | Annealed | 2 | 2 min. 18 secs | No insoluble matter present |
| 6 | Normal | 3 | 1 min. 43 secs | No insoluble matter present |
| 7 | Annealed | 3 | 2 min. 23 secs | No insoluble matter present |
| 8 | Normal | 4 | 1 min. 38 secs | No insoluble matter present |
| 9 | Annealed | 4 | 2 min. 31 secs | No insoluble matter present |
| 10 | Normal | 5 | 1 min. 39 secs | No insoluble matter present |
| 11 | Annealed | 5 | 2 min. 21 secs | No insoluble matter present |
| 12* | Normal | 5 | 3 min. 28 secs | No insoluble matter present |
| 13** | Normal | 5 | 5 min. 24 secs | No insoluble matter present |

*12 mL fill in 20 mL vial;
**24 mL fill in 50 mL vial

The results in Table 8 demonstrate a formulation and process which yield a product that will reconstitute in an acceptable amount of time. However, Samples 12 and 13, which reflect actual sample fill volumes, show that the total amount of product in a vial does affect the dissolution time. Glycine had a very minimal effect on dissolution time and that annealing seemed to increase dissolution time.

To further examine the dissolution time, the formulation concentration, vial size and fill, and glycine content were also examined. Samples were formulated to contain 150 mg/mL and 25 mg/mL zosuquidar. Glycine was also added to several of the samples to determine if there is an effect on the dissolution time. 16 mL aliquots were filled into either 20 mL×20 mm vials or 30 mL×20 mm vials, and were freeze-dried using a conservative cycle. The amount of added glycine, the vial size, the dissolution times, and a visual description of the samples after reconstitution are listed in Table 9. Samples were reconstituted with 16 mL of purified water. All vials contained yellow cakes, which were slightly shrunken and fractured.

TABLE 9

| | Glycine, | | | Dissolution | |
|---|---|---|---|---|---|
| Sample | mg/mL | Vial Size | pH | Time | Description |
| 1 | 0 | 20 mL | 1.57 | 1 min. 48 sec | No insoluble |
| | | | | | matter present |
| 2 | 0 | 30 mL | 1.57 | 1 min. 52 sec | No insoluble |
| | | | | | matter present |
| 3 | 5 | 20 mL | 2.37 | 1 min. 51 sec | No insoluble |
| | | | | | matter present |
| 4 | 5 | 30 mL | 2.37 | 1 min. 47 sec | No insoluble |
| | | | | | matter present |
| 5 | 20 | 20 mL | 3.19 | 1 min. 26 sec | No insoluble |
| | | | | | matter present |
| 6 | 20 | 30 mL | 3.19 | 1 min. 23 sec | No insoluble |
| | | | | | matter present |

These results show that the ratio and total solids amount in the samples tested produced cakes which dissolve in under 2 minutes, assuming an 800 mg dose delivered in 2 vials. Added glycine does not appear to affect the dissolution time. Glycine does however affect the pH of the formulation, which is problematic because previous studies have shown that as the pH increases the solubility of the API decreases. Because of these factors, it is desirable not to add glycine to the formulation.

The 25 mg/mL zosuquidar and 150 mg/mL CAPTISOL® formulation exhibits desirable formulation attributes; however, during lyophilization studies, it was observed that there was a small amount of undissolved “crust” stuck to the bottom of some of the samples after lyophilization and reconstitution. After watching samples during the freezing step in the lyophilization cycle, it was believed that CAPTISOL® was releasing the zosuquidar as the solution temperature decreased. The unbound zosuquidar would then precipitate and sink to the bottom of the vial where it would form a slowly dissolving crust. Upon reconstitution, most of the solids within the vial were completely dissolved in approximately 1 minute. The crust at the bottom of the vial on the other hand, would take several hours to completely dissolve.

Based on these results, a study was conducted to investigate the effects of varying the ratio of zosuquidar to CAPTISOL® and the amounts in an attempt to prevent zosuquidar from being released from the CAPTISOL® during freezing. Samples were prepared according to the concentrations listed in Table 10. 16 mL aliquots were filled into 30 mL×20 mm tubing vials and lyophilized using a conservative cycle. The 16 mL fill samples were reconstituted with 20 mL of purified water, and the 8 mL fill samples were reconstituted with 10 mL of purified water.

TABLE 10

| | Zosuquidar Free | | | |
|---|---|---|---|---|
| | Base | | CAPTISOL ® | |
| Vial # | Concentration | | Concentration | Solution Description after Reconstitution |
| 1 | 20 | mg/mL | 150 mg/mL | No crust or residue or free aggregates |
| 2 | 20 | mg/mL | 150 mg/mL | No crust or residue or free aggregates |
| 3 | 22.5 | mg/mL | 150 mg/mL | No crust or residue or free aggregates |
| 4 | 22.5 | mg/mL | 150 mg/mL | No crust or residue or free aggregates |
| 5 | 25 | mg/mL | 150 mg/mL | Slight crust present at bottom, no free |
| | | | | aggregates |
| 6 | 25 | mg/mL | 150 mg/mL | Slight crust present at bottom, no free |
| | | | | aggregates |
| 7* | 25 | mg/mL | 150 mg/mL | Slight crust present at bottom, no free |
| | | | | aggregates |
| 8* | 25 | mg/mL | 150 mg/mL | Slight crust present at bottom, no free |
| | | | | aggregates |
| 9* | 25 | mg/mL | 150 mg/mL | Slight crust present at bottom, no free |
| | | | | aggregates |
| 10* | 25 | mg/mL | 150 mg/mL | Slight crust present at bottom, no free |
| | | | | aggregates |
| 11 | 25 | mg/mL | 175 mg/mL | No crust or residue or free aggregates, slow |
| | | | | dissolution |
| 12 | 25 | mg/mL | 175 mg/mL | No crust or residue or free aggregates, slow |
| | | | | dissolution |
| 13 | 25 | mg/mL | 200 mg/mL | No crust or residue or free aggregates, slow |
| | | | | dissolution |
| 14 | 25 | mg/mL | 200 mg/mL | No crust or residue or free aggregates, slow |
| | | | | dissolution |
| 15 | 25 | mg/mL | 225 mg/mL | No crust or free aggregates, very difficult to |
| | | | | dissolve |
| 16 | 25 | mg/mL | 225 mg/mL | No crust or free aggregates, very difficult to |
| | | | | dissolve |

*8 mL fill in a 30 mL × 20 mm vial

Based on these results, the optimal concentration of CAPTISOL® and zosuquidar was 150 mg/mL (15%) and 22.5 mg/mL, respectively.

Three vials of zosuquidar (275 mg/vial zosuquidar, 1850 mg/vial CAPTISOL®) were each reconstituted with 15 mL of 5% Dextrose Injection, USP, 500 mL. A total of 47 mL of sample solution was removed from the vials with a 50 mL syringe and was injected into the 500 mL bag of 5% Dextrose Injection. The Zosuquidar/Dextrose solution was held at room temperature, and samples were removed at 0, 2, 4, 8, 12, 24, and 48 hours. All samples were held at −70° C. after being pulled and were analyzed after all samples had been collected. Each sample was tested for pH, HPLC concentration, and related substances. The data obtained from this study is summarized in Table 11. There was essentially no change in potency, impurities, and pH for all samples. Based on these results, zosuquidar with CAPTISOL® is stable at room temperature for 48 hours when reconstituted with 5% Dextrose Injection.

TABLE 11

| | | Zosuquidar Free Base |
|---|---|---|
| Time Point | pH | Conc. (mg/mL) |
| t = 0 hours | 2.63 | 1.41 |
| t = 2 hours | 2.62 | 1.41 |
| t = 4 hours | 2.64 | 1.41 |
| t = 8 hours | 2.63 | 1.42 |
| t = 12 hours | 2.63 | 1.40 |
| t = 24 hours | 2.64 | 1.40 |
| t = 48 hours | 2.64 | 1.40 |

Discussion

A drug product comprising 275 mg of zosuquidar trihydrochloride in CAPTISOL was formulated that exhibited superior solubility characteristics. Use of CAPTISOL® afforded over a 5-fold increase in water solubility of zosuquidar trihydrochloride, enabling lyophilization of a greater quantity of active ingredient in a 30 mL vial. To provide a dose of 275 mg/vial, a fill volume of 12.2 mL per 30 mL vial was employed. The total CAPTISOL® concentration per vial was 1.83 g. This concentration of CAPTISOL® solubilized zosuquidar and provided an acceptable reconstitution rate for the vial. Reconstitution of vials with 15 mL of 5% Dextrose Injection provided a solution that contained 16.9 mg/mL of zosuquidar.

Use of CAPTISOL® achieves a higher drug content per vial, an acceptable reconstitution time, and an acceptable lyophilized cake compared to other solubilizers such as mannitol and glycine, as demonstrated by the data in Table 12.

TABLE 12

| Parameter | Zosuquidar at 50 mg/vial | Zosuquidar at 275 mg/vial |
|---|---|---|
| Bulk Formulation | 5 mg/mL zosuquidar | 22.5 mg/mL zosuquidar |
| Concentration | 20 mg/mL mannitol | 150 mg/mL CAPTISOL ® |
| | 1.5 mg/mL glycine | |
| Fill volume per vial | 10 mL | 12.7 mL |
| Vial Content | 50 mg zosuquidar | 286 mg zosuquidar |
| | 200 mg mannitol | 1905 mg CAPTISOL ® |
| | 15 mg glycine | |
| Vial size | 30 mL Type 1 glass tubing vial | 30 mL Type 1 glass tubing vial |
| Appearance | Light yellow solid cake | Light yellow solid cake |
| Reconstitution time | Approx. 1 to 2 min | Approx 1 to 1.5 min |
| Reconstitution volume | 10 mL | 17.3 mL |
| Reconstitution | 5 mg/mL | 16.5 mg/mL |
| concentration | | |
| Number of vials | 11 | 2 |
| required per 550 mg/ | | |
| day dose | | |

Use of CAPTISOL® in combination with zosuquidar provides a stable formulation, as demonstrated by the real time stability data in Table 13.

TABLE 13

| Test | Initial | 1 month |
|---|---|---|
| Appearance (solid) | Pale light yellow solid | Light yellow solid |
| | cake | cake |
| Appearance (liquid) | Light yellow liquid | Light yellow liquid |
| pH | 1.68 | 1.81 |
| Assay by HPLC | 102.7% | 102.9% |
| Total Related substances by | 0.18% | 0.32% |
| HPLC | | |
| Moisture by KF | 1.0% | 1.0% |

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. A method of treating cancer in a patient exhibiting positive P-glycoprotein expression or positive P-glycoprotein function, the method comprising:
administering to the patient a chemotherapeutic agent that is a substrate for P-glycoprotein efflux and a stable chemotherapeutic composition comprising zosuquidar in combination with a modified cyclodextrin, whereby the cancer is treated.

2. The method of claim 1, wherein the modified cyclodextrin is a hydroxypropyl-β-cyclodextrin.

3. The method of claim 1, wherein the modified cyclodextrin is a sulfobutylcyclodextrin.

4. The method of claim 3, wherein the sulfobutylcyclodextrin is a polyanionic β-cyclodextrin derivative with a sodium sulfonate salt separated from a lipophilic cavity by a butyl ether spacer group.

5. The method of claim 3, wherein the stable chemotherapeutic composition is in lyophilized form.

6. The method of claim 3, wherein the stable chemotherapeutic composition is in solution form.

7. The method of claim 3, wherein the stable chemotherapeutic composition is in liquid unit dosage form, comprising from about 10 mg/mL to about 30 mg/mL zosuquidar and from about 100 mg/mL to about 200 mg/mL sulfobutylcyclodextrin.

8. The method of claim 3, wherein the stable chemotherapeutic composition is in liquid unit dosage form, comprising from about 20 mg/mL to about 25 mg/mL zosuquidar and from about 125 mg/mL to about 175 mg/mL sulfobutylcyclodextrin.

9. The method of claim 3, wherein the stable chemotherapeutic composition is in liquid unit dosage form, comprising about 22.5 mg/mL zosuquidar and about 150 mg/mL sulfobutylcyclodextrin.

10. The method of claim 3, wherein the stable chemotherapeutic composition is in lyophilized form, comprising zosuquidar and sulfobutylcyclodextrin in a weight ratio of zosuquidar to sulfobutylcyclodextrin of from about 1:5.7 to about 1:7.4.

11. The method of claim 3, wherein the stable chemotherapeutic composition is in lyophilized form, comprising zosuquidar and sulfobutylcyclodextrin in a weight ratio of zosuquidar to sulfobutylcyclodextrin of from about 1:6 to about 1:7.

12. The method of claim 3, wherein the stable chemotherapeutic composition is in lyophilized form, comprising zosuquidar and sulfobutylcyclodextrin in a weight ratio of zosuquidar to sulfobutylcyclodextrin of about 1:6.73.

13. The method of claim 3, wherein the stable chemotherapeutic composition is a dextrose solution.

14. The method of claim 3, wherein the cancer is acute myelogenous leukemia.

15. The method of claim 3, wherein the cancer is a carcinoma.

16. The method of claim 15, wherein the carcinoma is breast cancer.

17. The method of claim 15, wherein the carcinoma is ovarian cancer.

18. The method of claim 3, wherein the cancer is a sarcoma.

19. The method of claim 3, wherein the cancer is a hematologic malignancy.

20. The method of claim 19, wherein the hematologic malignancy is selected from the group consisting of acute lymphoblastic leukemia, chronic myeloid leukemia, plasma cell dyscrasias, lymphoma, and myelodysplasia.

21. The method of claim 3, wherein the chemotherapeutic agent is an anthracycline.

22. The method of claim 21, wherein the anthracycline is selected from the group consisting of doxorubicin, daunorubicin, epirubicin, idarubicin, and mitoxantrone.

23. The method of claim 3, wherein the chemotherapeutic agent is a Topoisomerase-II inhibitor.

24. The method of claim 23, wherein the Topoisomerase-II inhibitor is etoposide or teniposide.

25. The method of claim 3, wherein the chemotherapeutic agent is a vinca.

26. The method of claim 25, wherein the vinca is selected from the group consisting of vincristine, vinblastine, vinorelbine, and vindesine.

27. The method of claim 3, wherein the chemotherapeutic agent is a taxane.

28. The method of claim 27, wherein the taxane is paclitaxel or docetaxel.

29. The method of claim 3, wherein the chemotherapeutic agent is selected from the group consisting of gleevec, dactinomycin, bisantrene, mitoxantrone, actinomyocin D, mithomycin C, mitramycin, methotrexate, adriamycin, mitomycin, and mithramycin, anthracene, and epipodophyllo-toxin.

30. The method of claim 3, wherein the chemotherapeutic agent comprises daunorubicin and cytarabine, and wherein the cancer is newly diagnosed acute myelogenous leukemia.

31. The method of claim 3, wherein the chemotherapeutic agent comprises Mylotarg, and wherein the cancer is relapsed acute myelogenous leukemia.
